(19) United States
(12) Patent Application Publication
(10) Pub. No.: US 2005/0137527 A1
(43) Pub. Date: Jun. 23, 2005

(54) Graduated sheath and dilator assembly

(75) Inventor: David Kunin, North Wales, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/019,851

(22) Filed: Dec. 22, 2004

Related U.S. Application Data
(60) Provisional application No. 60/531,948, filed on Dec. 23, 2003.

(51) Int. Cl. A61M 29/00

(52) U.S. Cl. 604/104

(57) ABSTRACT

A sheath and a dilator assembly is disclosed. The assembly includes a sheath having a proximal sheath end, a distal sheath end, and a generally tubular sheath body extending between the proximal sheath end and the distal sheath end. The sheath body includes sheath indicator indicia extending from the distal sheath end toward the proximal sheath end. A dilator is disposed within the sheath. The dilator includes a proximal dilator end extending proximally of the proximal sheath end, a distal dilator end extending distally of the distal sheath end, and a dilator body extending between the proximal dilator end and the distal dilator end. The dilator body includes dilator indicator indicia extending from the distal dilator end toward the proximal dilator end, such that the sheath indicator indicia and the dilator indicator indicia display predetermined dimensions from the distal dilator end toward the proximal dilator end.

### CROSS-REFERENCE TO RELATED APPLICATION

The present application claims the benefit of U.S. Provisional Patent Application Ser. No. 60/531,948, filed 23 Dec. 2003.

### FIELD OF THE INVENTION

The present invention relates to a dilator and sheath assembly that is used during the insertion of a catheter into a patient's blood vessel.

### BACKGROUND OF THE INVENTION

Catheters are often used for the delivery and withdrawal of fluids to and from a blood vessel in a patient, respectively. The fluids may be medication that is administered to the patient, or blood that is withdrawn from the patient.

Typically, to insert a catheter into a blood vessel, the blood vessel is located by known methods. An aspirating needle is inserted into the vessel to confirm placement within the vessel. A guide wire is then inserted through a proximal end of the aspirating needle and into the vessel. The aspirating needle is withdrawn by sliding the needle proximally over the guide wire, leaving the guide wire within the vessel. If a catheter with a sufficiently hard wall is being used, the catheter may be slid over the guide wire, directly into the vessel.

However, for some catheters, particularly soft walled catheters, a dilator is required to dilate the vessel at the insertion point in order to accommodate the insertion of the catheter. The dilator is typically inserted into a sheath and initially used as a dilator and sheath assembly. The assembly is inserted into the vessel over the guide wire and the dilator is used to dilate the insertion opening in the vessel wall. After the insertion opening is dilated, the dilator and the guide wire are removed from the vessel by removing both the dilator and the guide wire proximally from the sheath. The sheath remains in the vessel to accommodate insertion of the catheter through the sheath and into the blood vessel. After the catheter is inserted into the blood vessel, the sheath is torn from around the catheter and removed from the vessel.

However, once distal ends of the dilator and sheath assembly are inserted through the patient's skin and into the vessel, it is impossible to tell exactly how far the dilator has been inserted, running the risk of the inserting physician inserting the dilator too far and damaging the vessel walls. It would be beneficial to provide an ability for the inserting physician to determine exactly how far the dilator has been inserted into the patient.

### BRIEF SUMMARY OF THE INVENTION

Briefly, the present invention provides a sheath and a dilator assembly. The assembly comprises a sheath having a proximal sheath end, a distal sheath end, and a generally tubular sheath body extending between the proximal sheath end and the distal sheath end. The sheath body includes sheath indicator indicia extending from the distal sheath end toward the proximal sheath end. The assembly also includes a dilator disposed within the sheath. The dilator includes a proximal dilator end extending proximally of the proximal sheath end, a distal dilator end extending distally of the distal sheath end, and a dilator body extending between the proximal dilator end and the distal dilator end. The dilator body includes dilator indicator indicia extending from the distal dilator etid toward the proximal dilator end, such that the sheath indicator indicia and the dilator indicator indicia display predetermined dimensions from the distal dilator end toward the proximal dilator end.

Additionally, the present invention also provides sheath and dilator assembly comprising a sheath having a proximal sheath end, a distal sheath end, and a generally tubular sheath body extending between the proximal sheath end and the distal sheath end. The sheath body includes sheath indicator indicia extending from the distal sheath end toward the proximal sheath end. A first connecting means is disposed at the proximal sheath end. A dilator is disposed within the tubular sheath body. The dilator includes a proximal dilator end extending proximally of the proximal sheath end, a distal dilator end extending distally of the distal sheath end, and a dilator body extending between the proximal dilator end and the distal dilator end. The dilator body includes dilator indicator indicia extending from the distal dilator end toward the proximal dilator end, such that the sheath indicator indicia and the dilator indicator indicia display predetermined dimensions from the distal dilator end toward the proximal sheath end. A second connecting means is disposed at the proximal dilator end. The first and second connecting means are releasably engageable with each other to releasably secure the dilator within the sheath.

The present invention also provides a method of inserting a sheath and dilator assembly into a patient's blood vessel. The sheath and dilator assembly comprise a sheath having a proximal sheath end, a distal sheath end, and a generally tubular sheath body extending between the proximal sheath end and the distal sheath end. The sheath body includes sheath indicator indicia extending from the distal sheath end toward the proximal sheath end. The assembly also includes a dilator disposed within the sheath. The dilator includes a proximal dilator end extending proximally of the proximal sheath end, a distal dilator end extending distally of the distal sheath end, and a dilator body extending between the proximal dilator end and the distal dilator end. The dilator body includes dilator indicator indicia extending from the distal dilator end toward the proximal dilator end, such that the sheath indicator indicia and the dilator indicator indicia display predetermined dimensions from the distal dilator end toward the proximal dilator end. The method furher includes inserting the distal dilator end and the distal sheath end into the blood vessel a distance determined by the sheath indicator indicia and the dilator indicator indicia.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a top plan view of a dilator and sheath assembly according to a preferred embodiment of the present invention.

FIG. 2 is an exploded top plan view of the dilator and sheath assembly of FIG. 1.

FIG. 3 shows a partially cutaway view of the dilator and sheath assembly being inserted into a patient's blood vessel.

### DETAILED DESCRIPTION OF THE INVENTION

In the drawings, like numerals indicate like elements throughout. Certain terminology is used herein for convenience only and is not to be taken as a limitation on the present invention. The words “proximal” and “distal” refer to directions away from and closer to, respectively, the insertion tip of the dilator in the dilator and sheath assembly according to the present invention. The terminology includes the words above specifically mentioned, derivatives thereof, and words of similar import. The following describes a preferred embodiment of the invention. However, it should be understood based on this disclosure, that the invention is not limited by the preferred embodiment described herein.

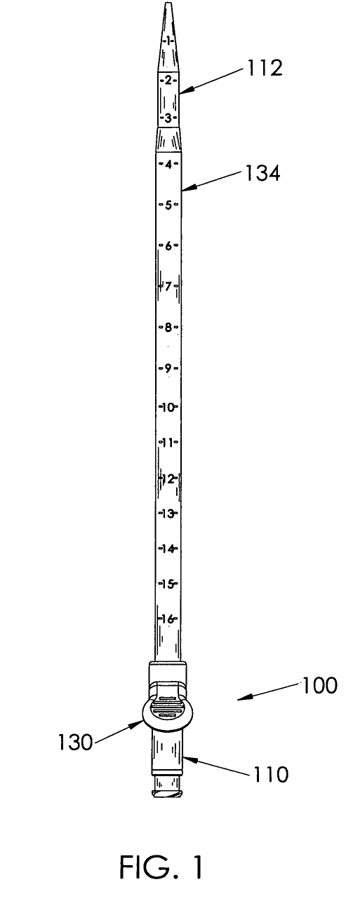

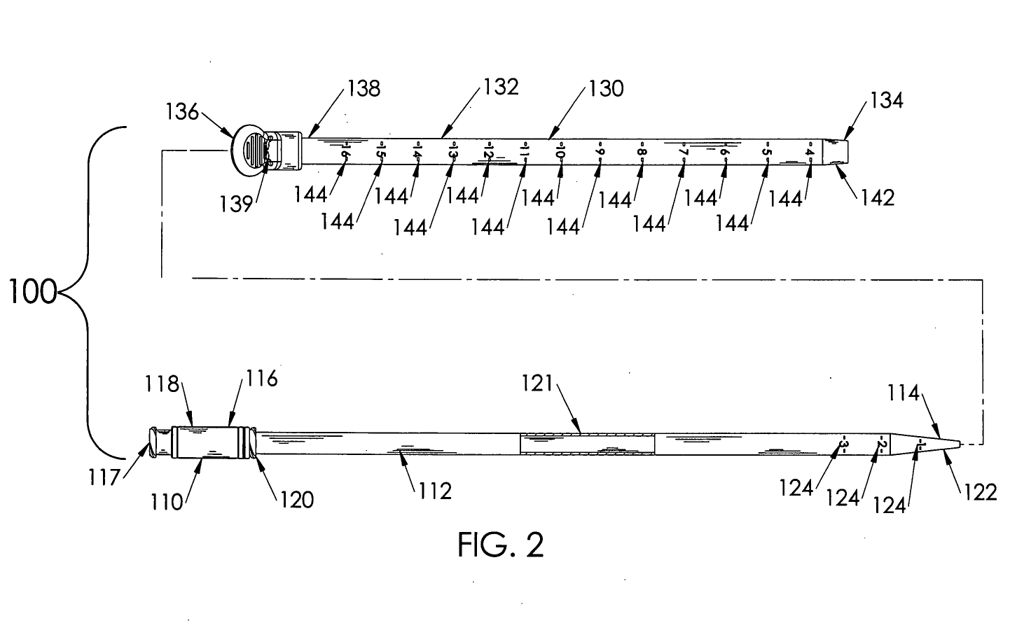

Referring now to FIGS. 1 and 2, a dilator and sheath assembly 100 according to the present invention is shown. The assembly 100 includes a dilator 110 that is disposable within a sheath 130. The dilator 110 includes a hollow elongated body 112 that has a tapered distal tip 114 and a connector 116 at a proximal end 118. Preferably, the connector 116 is a luer connector, or some other suitable connector. The connector 116 preferably includes male threads 117 to engage mating female threads on a medical device (not shown) that may be releasably connected to the connector 116. The connector 116 preferably includes locking threads 120 at a distal end of the connector 116.

A hollow passage 121 extends through the body 112 between the distal tip 114 and the proximal end 118. The hollow passage 121 is sufficiently sized to allow the dilator 110 to be inserted over a guide wire (not shown) such that the guide wire passes through the hollow passage 121.

A distal end 122 of the dilator 110 includes indicator indicia 124 that extend toward the proximal end 118 of the dilator 110. Preferably, the indicator indicia 124 include markings to indicate distance from the distal tip 114. The distance may be in centimeters or other predetermined distances. Also preferably, the indicator indicia 124 include numerical references to further indicate distance. The indicator indicia 124 may extend entirely around an outer circumference of the body 112, or the indicator indicia 124 may only extend partially around the outer circumference of the body 112.

The sheath 130 includes a hollow elongated body 132 that has a tapered distal tip 134 and a handle 136 at a proximal end 138. Preferably, the handle 136 includes mating female threads 139 disposed to releasably engage the locking threads 120 on the dilator 110 when the dilator 110 is inserted into the sheath 130. The female threads 139 and the locking threads 120 provide for a releasable connection between the dilator 110 and the sheath 130 after the dilator 110 is inserted into the sheath 130. The dilator 110 can then be rotated approximately 90 degrees so that the locking threads 120 are releasably engaged with the female threads 139.

A distal end 142 of the sheath 130 includes indicator indicia 144 that extend toward the proximal end 138 of the sheath 130. Preferably, the indicator indicia 144 include markings to indicate distance from the distal tip 14 of the dilator 110 when the dilator 110 is fully inserted into the sheath 130. The distance is preferably in the same units as the distance on the dilator 110 as described above. Also preferably, the indicator indicia 144 include numerical references to further indicate distance. The indicator indicia 144 may extend entirely around an outer circumference of the body 132, or the indicator indicia 144 may only extend partially around the outer circumference of the body 132.

Preferably, the dilator 110 and the sheath 130 are constructed from a radiopaque polymer such as polypropylene, polyethylene, TEFLON®, or other suitable biocompatible, with BaSO₄ or other suitable material added for radiopacity. The indicator indicia 124, 144 may be radiopaque or non-radiopaque. Alternatively, the dilator 110 and the sheath 130 may be constructed from a virgin, non-radiopaque polymer such as polypropylene, polyethylene, TEFLON®, with the indicator indicia 124, 144 being radiopaque.

Initially, the dilator 110 is preferably fuilly inserted into the sheath 130 as seen in FIG. 1. The distal tip 134 of the sheath 130 tapers such that the dilator body 112 at the distal tip 134 of the sheath 130 frictionally engages the distal tip 134 of the sheath 130. The taper of the distal tip 134 of the sheath 130 provides a smooth transition between the dilator 110 and the sheath 130. The locking threads 120 are engaged with the female threads 139, securely retaining the dilator 110 within the sheath 130.

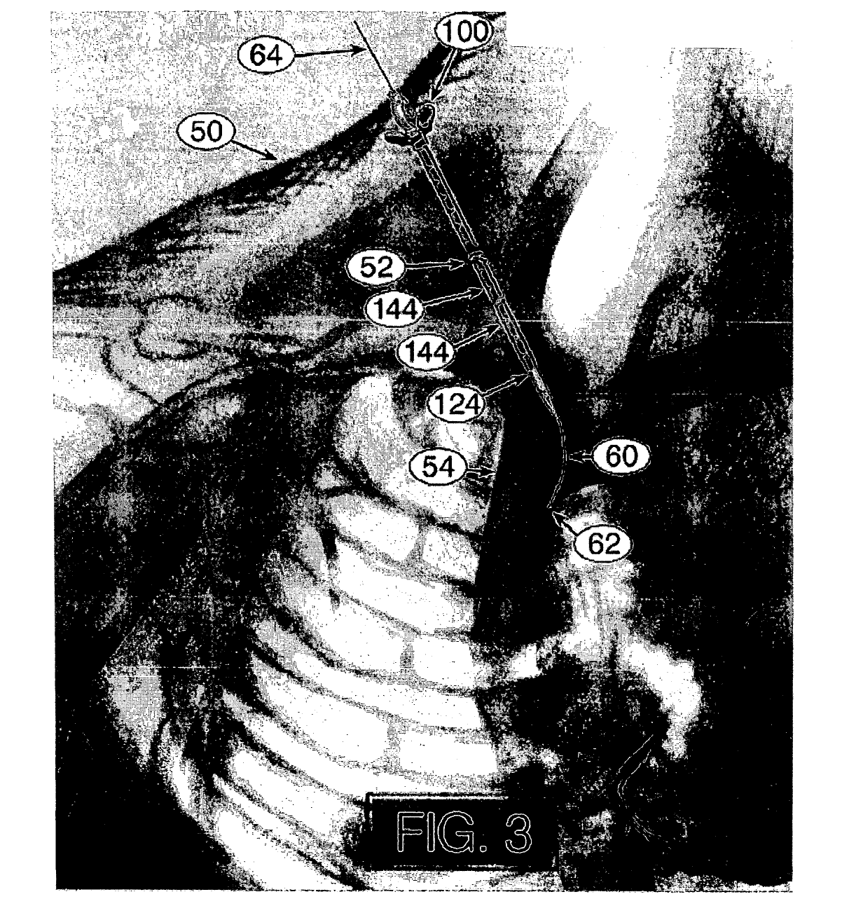

Referring now to FIG. 3, to use the dilator and sheath assembly 100 during insertion of a catheter (not shown) in a patient 50, an inserting physician locates an incision point according to known techniques and makes an appropriate incision 52 in the patient 50. An aspirating needle with syringe (not shown) is inserted into the incision 52 and an appropriate blood vessel 54, such as the internal jugular vein, is located according to known techniques. The syringe is removed and a distal end 62 of a guide wire 60 is inserted through the needle and into the vessel 54. The needle is then removed, leaving the guide wire 60 in place.

The physician inserts a proximal end 64 of the guide wire 60 that is exposed into the hollow passage 121 of the dilator 110 and slides the dilator and sheath assembly 100 over the guide wire 60 so that the distal tip 114 of the dilator 110 enters the incision 52. The physician continues to insert the dilator 110 and subsequently, the sheath 130, into the incision 52, while watching the indicator indicia 124, 144 on the dilator 110 and sheath 130. By way of example only, the indicator indicia 124, 144 shown in FIG. 3 represent one centimeter spacings between adjacent indicia 124, 144. When the physician has determined that the dilator and sheath assembly 100 has been sufficiently inserted into the patient 50, as indicated by the indicator indicia 124, 144, the physician stops inserting the dilator and sheath assembly 100 into the patient 50. As shown in FIG. 3, the dilator and sheath assembly 100 has been inserted approximately 10 centimeters into the patient 50. At this point, the guide wire 60 may be removed from the patient by grasping the proximal end 64 of the guide wire 60 and pulling the guide wire 60 from the vessel 52.

The physician then rotates the dilator 110 approximately 90 degrees relative to the sheath 130 to unlock the locking threads 120 from the female threads 139. The dilator 110 is then pulled from the proximal end 138 of the sheath 130 and removed. A catheter (not shown) is then inserted into the incision 52 through the sheath 130 according to known methods. After the catheter is fully inserted, the physician grasps the handle 136 of the sheath 130 and tears the sheath 130, while pulling the sheath 130 from the patient 50.

An example of a dilator and sheath assembly 100 that may be used with the present invention is disclosed in U.S. Pat. No. 6,796,991 (“the '991 patent”), which issued on Sep. 28, 2004, and is owned by the assignee of the present invention and is incorporated herein as though fully set forth.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A sheath and a dilator assembly comprising:
a sheath having a proximal sheath end, a distal sheath end, and a generally tubular sheath body extending between the proximal sheath end and the distal sheath end,
wherein the sheath body includes sheath indicator indicia extending from the distal sheath end toward the proximal sheath end; and
a dilator disposed within the sheath, wherein the dilator includes a proximal dilator end extending proximally of the proximal sheath end, a distal dilator end extending distally of the distal sheath end, and a dilator body extending between the proximal dilator end and the distal dilator end, wherein the dilator body includes dilator indicator indicia extending from the distal dilator end toward the proximal dilator end, such that the sheath indicator indicia and the dilator indicator indicia display predetermined dimensions from the distal dilator end toward the proximal dilator end.

2. The sheath and dilator assembly according to claim 1, wherein at least one of the sheath and the dilator is radiopaque.

3. The sheath and dilator assembly according to claim 1, wherein at least one of the sheath indicator indicia and the dilator indicator indicia is radiopaque.

4. The sheath and dilator assembly according to claim 1, wherein the dilator indicator indicia extends between the distal dilator end and the distal sheath end.

5. The sheath and dilator assembly according to claim 1, wherein the predetermined dimensions are centimeters.

6. The sheath and dilator assembly according to claim 1, wherein the proximal sheath end comprises at least one female thread disposed at a proximal end of the sheath body and wherein the proximal dilator end comprises at least one locking thread adapted to releasably engage the at least one female thread to releasably secure the dilator and the sheath.

7. A sheath and dilator assembly comprising:
a sheath having:
a proximal sheath end;
a distal sheath end;
a generally tubular sheath body extending between the proximal sheath end and the distal sheath end, wherein the sheath body includes sheath indicator indicia extending from the distal sheath end toward the proximal sheath end; and
a first connecting means disposed at the proximal sheath end; and
a dilator disposed within the tubular sheath body, wherein the dilator includes:
a proximal dilator end extending proximally of the proximal sheath end;
a distal dilator end extending distally of the distal sheath end;
a dilator body extending between the proximal dilator end and the distal dilator end, wherein the dilator body includes dilator indicator indicia extending from the distal dilator end toward the proximal dilator end, such that the sheath indicator indicia and the dilator indicator indicia display predetermined dimensions from the distal dilator end toward the proximal sheath end; and
a second connecting means disposed at the proximal dilator end, wherein the first and second connecting means are releasably engageable with each other to releasably secure the dilator within the sheath.

8. The sheath and dilator assembly according to claim 7, wherein at least one of the sheath and the dilator is radiopaque.

9. The sheath and dilator assembly according to claim 7, wherein at least one of the sheath indicator indicia and the dilator indicator indicia is radiopaque.

10. The sheath and dilator assembly according to claim 7, wherein the dilator indicator indicia extends between the distal dilator end and the distal sheath end.

11. The sheath and dilator assembly according to claim 7, wherein the predetermined dimensions are centimeters.

12. The sheath and dilator assembly according to claim 1, wherein the first connecting means comprises at least one female thread disposed at a proximal end of the sheath body and wherein the second connecting means comprises at least one locking thread adapted to releasably engage the at least one female thread to releasably secure the dilator and the sheath.

13. A method of inserting a sheath and dilator assembly into a patient's blood vessel, wherein the sheath and dilator assembly comprise:
a sheath having a proximal sheath end, a distal sheath end, and a generally tubular sheath body extending between the proximal sheath end and the distal sheath end, wherein the sheath body includes sheath indicator indicia extending from the distal sheath end toward the proximal sheath end; and
a dilator disposed within the sheath, wherein the dilator includes a proximal dilator end extending proximally of the proximal sheath end, a distal dilator end extending distally of the distal sheath end, and a dilator body extending between the proximal dilator end and the distal dilator end, wherein the dilator body includes dilator indicator indicia extending from the distal dilator end toward the proximal dilator end, such that the sheath indicator indicia and the dilator indicator indicia display predetermined dimensions from the distal dilator end toward the proximal dilator end;
wherein the method coi nprises inserting the distal dilator end and the distal sheath end into the blood vessel a distance determined by the sheath indicator indicia and the dilator indicator indicia.

14. The method according to claim 13, further comprising, after inserting the distal dilator end and the distal sheath end into the blood vessel, rotating the dilator relative to the sheath.

15. The method according to claim 14, further comprising, after rotating the dilator relative to the sheath, displacing the dilator proximally relative to the sheath.
